(19) United States
(12) Patent Application Publication
(10) Pub. No.: US 2014/0045883 A1
(43) Pub. Date: Feb. 13, 2014

(54) ACK1 KINASE INHIBITION TO TREAT TRIPLE NEGATIVE BREAST CANCER

(71) Applicants: Nupam P. Mahajan, Tampa, FL (US); Kiran N. Mahajan, Tampa, FL (US)

(72) Inventors: Nupam P. Mahajan, Tampa, FL (US); Kiran N. Mahajan, Tampa, FL (US)

(73) Assignee: H. Lee Moffitt Cancer Center and Research Institute, Inc., Tampa, FL (US)

(21) Appl. No.: 13/965,034

(22) Filed: Aug. 12, 2013

Related U.S. Application Data
(60) Provisional application No. 61/682,537, filed on Aug. 13, 2012.

(51) Int. Cl. A61K 31/437; G01N 33/573

(52) U.S. Cl. CPC A61K 31/437; G01N 33/573
USPC 514/300; 506/9; 435/7.4

(57) ABSTRACT

Compositions and methods are disclosed for treating Triple Negative Breast Cancers (TNBCs). The methods involve administering to a subject with TNBC a composition containing an Ack1 kinase inhibitor. In some embodiments, the method involves first assaying a sample from the subject for Tyr176-phosphorylated-AKT and/or Tyr284-phosphorylated-Ack1. In these embodiments, detection of the phosphorylated AKT and/or Ack1 is an indication that the subject is a suitable candidate for treatment with the Ack1 kinase inhibitor.

### CROSS-REFERENCE TO RELATED PATENT APPLICATION

This application claims priority under 35 U.S.C. §119(e) to U.S. Provisional Application No. 61/682,537, filed Aug. 13, 2012, the content of which is incorporated herein by reference in its entirety.

### BACKGROUND OF THE INVENTION

Triple Negative Breast Cancers (TNBCs), representing about 15% of all breast cancers, are highly aggressive type of tumors that lack estrogen receptor (ER), progesterone receptor (PR) and ERBB2 (HER2) gene amplification (Elias A D. American journal of clinical oncology. 2010 33(6):637-45). TNBCs affect more frequently younger patients, and are more prevalent in African-American women (Morris G J, et al. Cancer. 2007 110(4):876-84). These are large tumors of higher grade and often have lymph node involvement at diagnosis (Haffty B G, et al. Journal of clinical oncology. 2006 24(36):5652-7). Unfortunately, TNBCs patients have a higher rate of distant recurrence and a poorer prognosis than women with other breast cancer subtypes (Haffty B G, et al. Journal of clinical oncology. 2006 24(36):5652-7; Dent R, et al. Clinical cancer research. 2007 13(15 Pt 1):4429-34). Less than 30% of women with metastatic TNBCs survive 5 years, and almost all die of their disease despite adjuvant chemotherapy (Dent R, et al. Clinical cancer research. 2007 13(15 Pt 1):4429-34). While significant advances have been made for personalized therapy for ER and HER2-positive breast cancer patients, there are a few biomarkers currently available for TNBC patients and targeted therapeutic options for women with TNBCs remain practically non-existent.

### SUMMARY OF THE INVENTION

Compositions and methods are disclosed for treating Triple Negative Breast Cancers (TNBCs). The methods involve administering to a subject with TNBC a composition containing an Ack1 kinase inhibitor. In some embodiments, the method involves first assaying a sample from the subject for Tyr176-phosphorylated-AKT and/or Tyr284-phosphorylated-Ack1. In these embodiments, detection of the phosphorylated AKT and/or Ack1 is an indication that the subject is a suitable candidate for treatment with the Ack1 kinase inhibitor.

Provided herein is a method for treating Triple Negative Breast Cancer (TNBC) in a subject, comprising administering to the subject a composition comprising a Ack1 kinase inhibitor. Also provided is a method for selecting a therapy for a subject with Triple Negative Breast Cancer (TNBC), comprising

(a) assaying a sample from the subject for expression level of Tyrosine 176-phosphorylated-AKT, Tyrosine 284-phosphorylated-Ack1, or a combination thereof; and

(b) comparing the expression level to a control level;

wherein detection of an elevated expression of Tyrosine 176-phosphorylated-AKT, Tyrosine 284-phosphorylated-Ack1, or a combination thereof, compared to the control is an indication that an Ack1 kinase inhibitor is selected as the therapy for treating the subject.

Yet further provided is a method for treating a subject with Triple Negative Breast Cancer (TNBC), comprising

(a) assaying a sample from the subject for expression level of Tyrosine 176-phosphorylated-AKT, Tyrosine 284-phosphorylated-Ack1, or a combination thereof;

(b) detecting elevated expression of Tyrosine 176-phosphorylated-AKT, Tyrosine 284-phosphorylated-Ack1, or a combination thereof, compared to a control level; and

(c) administering to the subject a composition comprising a Ack1 kinase inhibitor.

In one aspect of the above methods, the Ack1 kinase inhibitor is defined by Formula I

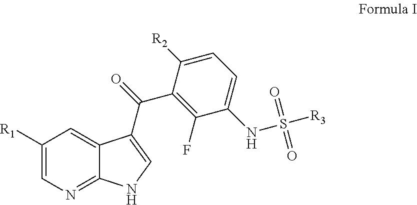

or a pharmaceutically acceptable salt or prodrug thereof,
wherein
R₁ is selected from the group consisting of hydrogen, halogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —OH, —NH₂, —CN, —NO₂, —C(O)OH, —S(O)₂NH₂, —C(O)NH₂, —C(S)NH₂, —NHC(O)NH₂, —NHC(S)NH₂, —NHS(O)₂NH₂, —OR₄, —SR₄, —NR₄R₅, —C(O)R₄, —C(S)R₄, —C(O)OR₄, —C(O)NR₄R₅, —C(S)NR₄R₅, —S(O)₂NR₄R₅, —NR₅C(O)R₄, —NR₅C(S)R₄, —NR₅S(O)₂R₄, —NR₅C(O)NH₂, —NR₅C(O)NR₅R₄, —NR₅C(S)NH₂, —NR₅C(S)NR₅R₄, —NR₅S(O)₂NH₂, —NR₅S(O)₂NR₅R₄, —S(O)R₄, and —S(O)₂R₄;
R₂ is selected from the group consisting of hydrogen, fluoro and chloro;
R₃ is selected from the group consisting of optionally substituted C₂-C₆ alkyl, optionally substituted aryl, optionally substituted heteroaryl, and —NR₆R₇;
R₄ is selected from the group consisting of optionally substituted lower alkyl, optionally substituted lower alkenyl, provided, however, that when R₄ is optionally substituted lower alkenyl, no alkene carbon thereof is bound to N, S, O, S(O), S(O)₂, C(O) or C(S) of —OR₄, —SR₄, —NR₄R₅, —C(O)R₄, —C(S)R₄, —C(O)OR₄, —C(O)NR₄R₅, —C(S)NR₄R₅, —S(O)₂NR₄R₅, —NR₅C(O)R₄, —NR₅C(S)R₄, —NR₅S(O)₂R₄, —NR₅C(O)NH₂, —NR₅C(O)NR₅R₄, —NR₅C(S)NH₂, —NR₅C(S)NR₅R₄, —NR₅S(O)₂NH₂, —NR₅S(O)₂NR₅R₄, —S(O)R₄, or —S(O)₂R₄, optionally substituted lower alkynyl, provided, however, that when R₄ is optionally substituted lower alkynyl, no alkyne carbon thereof is bound to N, S, O, S(O), S(O)₂, C(O) or C(S) of —OR₄, —SR₄, —NR₄R₅, —C(O) R₄, —C(S)R₄, —C(O)OR₄, —C(O)NR₄R₅, —C(S)NR₄R₅, —S(O)₂NR₄R₅, —NR₅C(O)R₄, —NR₅C(S)R₄, —NR₅S(O)₂R₄, —NR₅C(O)NH₂, —NR₅C(O)NR₅R₄, —NR₅C(S)NH₂, —NR₅C(S)NR₅R₄, —NR₅S(O)₂NH₂, —NR₅S(O)₂NR₅R₄, —S(O)R₄, or —S(O)₂R₄, optionally substituted cycloalkyl, optionally substituted heterocycloalkyl, optionally substituted aryl, and optionally substituted heteroaryl;
R₅ is selected from the group consisting of hydrogen and optionally substituted lower alkyl; and
R₆ and R₇ are independently hydrogen or optionally substituted lower alkyl, or R₆ and R₇, in combination with the nitrogen to which they are attached, form an optionally substituted 5-7 membered heterocycloalkyl. In a further aspect, the Ack1 kinase inhibitor is Vemurafenib.

### BRIEF DESCRIPTION OF THE DRAWINGS

FIG. 1A are images of Tissue Microarray (TMA) sections representing different breast cancer stages stained with pTyr284-Ack1 and pTyr176-AKT antibodies. FIG. 1B are box plots to summarize distributions of staining intensities for pTyr284-Ack1 in different stages of breast cancer. FIG. 1C are box plots to summarize distributions of staining intensities for pTyr176-AKT in different stages of breast cancer. FIG. 1D is a Kaplan-Meier analysis showing that individuals with breast cancer that have moderate to strong staining (>4) of pTyr284-Ack1 have a lower probability of survival (log rank test, p=0.08). FIG. 1E is a Kaplan-Meier analysis of the breast cancer patients that have moderate to strong staining (>4) of pTyr176-AKT.

FIG. 2 is an image of immunoblots showing that AIM-100 and PLX-4032 suppressed Ack1 activation in vivo. HEK293 cells were transfected, depleted of serum, untreated or treated with AIM-100 or PLX4032 and following day cells were harvested. Immunoprecipitation with HA-beads followed by immunoblotting with pTyr (top and middle panel) or Ack1 (bottom panel) antibodies was performed.

FIGS. 3A-3B are images of immunoblots of lysates from serum starved MCF-7 and A2780-CP cells treated (or untreated) with insulin or EGF (10 ng/ml) and AIM-100 (3 μM, overnight) that were immunoblotted with pTyr176-AKT (top panel), pSer473-AKT (2nd panel), pThr308-AKT (3rd panel), pTyr284-Ack1 (4th panel) and tubulin (bottom panel).

FIG. 4 is a graph showing results of MTT assay for MCF-7, MDA-MB-468 (triangles) and MDA-MB-231 (circles) cell lines that were treated with increasing concentrations of PLX4032 (open) or AIM-100 (closed) for 72 hours. Experiment was performed twice with 8 replicates, a representative data set is shown.

FIG. 5 is a bar graph showing results of an ELISA for pTyr176-AKT detection after purified Ack1 was incubated with AKT peptide with or without Ack1 inhibitors AIM-100 or PLX4032 for 60 min at 30° C. Ack1 inhibitors AIM-100 and PLX4032 significantly reduced AKT activation.

FIG. 6 is a bar graph showing Affinity Purification coupled ELISA for pTyr176-AKT detection in TNBC derived cell line MDA-MB-231. MDA-MB-231 cell lysates were incubated with streptavidin beads with AKT peptide. The AKT peptides were eluted and phosphorylation was detected using ELISA. Ack1 inhibitors significantly reduced AKT activation.

### DETAILED DESCRIPTION

Triple Negative Breast Cancers (TNBCs) do not respond to hormonal therapy such as tamoxifen or aromatase inhibitors or therapies that target HER2 receptors, such as Herceptin (trastuzumab). Because of limited targets that are available for TNBCs, currently there is an intense interest in finding new targets and thus personalized medications that can treat this type of breast cancer. Using 28 different TNBC derived cell lines Ack1 was discovered to be hyperphosphorylated and thus activated in TNBCs. Further, both Basal-like TNBC cell line e.g. MDA-MB-468 and mesenchymal-like TNBC cell line e.g. MDA-MB-231, were shown to be sensitive to PLX-4032. These data open up a potential treatment option for those women that have Ack1-positive TNBC.

Analysis of the invasiveness and anchorage-independent growth of 28 TNBC cell lines followed by quantitative phosphotyrosine profiling revealed that Ack1 tyrosine kinase is hyperphosphorylated in TNBCs. Protein kinase AKT plays a central role in regulating growth and survival and is highly activated in human cancers including in TNBCs. Activated Ack1 directly phosphorylates AKT at an evolutionarily conserved tyrosine176 residue leading to AKT activation. Notably, levels of activated Ack1 (pY284-Ack1) and activated AKT (pY176-AKT) are significantly elevated in human primary breast tumors which correlated with severity of disease and inversely correlated with survival of patients.

AIM-100, a Ack1-specific small molecule kinase inhibitor, suppresses Ack1/AKT signaling in breast and prostate tumor derived cell lines and xenograst tumor growth. Significantly, Ack1 mediated AKT activation is unaffected by PI3K inhibitors. The FDA approved bRaf inhibitor, PLX4032 (Vemurafenib), is an excellent Ack1 inhibitor with IC₅₀ of 19 nM.

Therefore, compositions and methods are disclosed for treating Triple Negative Breast Cancers (TNBCs). The methods involve administering to a subject with TNBC a composition containing an Ack1 kinase inhibitor.

A variety of Ack1 kinase inhibitors may be administered to treat cancer, preferably Triple Negative Breast Cancers (TNBCs). Suitable Ack1 kinase inhibitors are known in the art. See, for example, U.S. Pat. Nos. 7,504,509 and 7,863,288 to Ibrahim, et al., which are incorporated herein by reference herein for their description of Ack1 kinase inhibitors. In one embodiment, the Ack1 kinase inhibitor is defined by Formula I

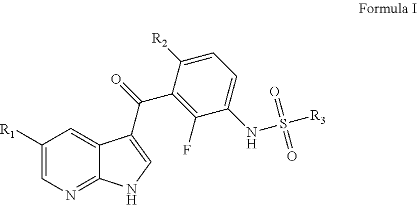

or a pharmaceutically acceptable salt or prodrug thereof,
wherein

R₁ is selected from the group consisting of hydrogen, halogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —OH, —NH₂, —CN, —NO₂, —C(O)OH, —S(O)₂NH₂, —C(O)NH₂, —C(S)NH₂, —NHC(O)NH₂, —NHC(S)NH₂, —NHS(O)₂NH₂, —OR₄, —SR₄, —NR₄R₅, —C(O)R₄, —C(S)R₄, —C(O)OR₄, —C(O)NR₄R₅, —C(S)NR₄R₅, —S(O)₂NR₄R₅, —NR₅C(O)R₄, —NR₅C(S)R₄, —NR₅S(O)₂R₄, —NR₅C(O)NH₂, —NR₅C(O)NR₅R₄, —NR₅C(S)NH₂, —NR₅C(S)NR₅R₄, —NR₅S(O)₂NH₂, —NR₅S(O)₂NR₅R₄, —S(O)R₄, and —S(O)₂R₄;

R₂ is selected from the group consisting of hydrogen, fluoro and chloro;

R₃ is selected from the group consisting of optionally substituted C₂-C₆ alkyl, optionally substituted aryl, optionally substituted heteroaryl, and —NR₆R₇;

R₄ is selected from the group consisting of optionally substituted lower alkyl, optionally substituted lower alkenyl, provided, however, that when R₄ is optionally substituted lower alkenyl, no alkene carbon thereof is bound to N, S, O, S(O), S(O)₂, C(O) or C(S) of —OR₄, —SR₄, —NR₄R₅, —C(O)R₄, —C(S)R₄, —C(O)OR₄, —C(O)NR₄R₅, —C(S)NR₄R₅, —S(O)₂NR₄R₅, —NR₅C(O)R₄, —NR₅C(S)R₄, —NR₅S(O)₂R₄, —NR₅C(O)NH₂, —NR₅C(O)NR₅R₄, —NR₅C(S)NH₂, —NR₅C(S)NR₅R₄, NR₅S(O)₂NH₂, —NR₅S(O)₂NR₅R₄, —S(O)R₄, or —S(O)₂R₄, optionally substituted lower alkynyl, provided, however, that when R₄ is optionally substituted lower alkynyl, no alkyne carbon thereof is bound to N, S, O, S(O), S(O)₂, C(O) or C(S) of —OR₄, —SR₄, —NR₄R₅, —C(O)R₄, —C(S)R₄, —C(O)OR₄, —C(O)NR₄R₅, —C(S)NR₄R₅, —S(O)₂NR₄R₅, —NR₅C(O)R₄, —NR₅C(S)R₄, —NR₅S(O)₂R₄, —NR₅C(O)NH₂, —NR₅C(O)NR₅R₄, —NR₅C(S)NH₂, —NR₅C(S)NR₅R₄, —NR₅S(O)₂NH₂, —NR₅S(O)₂NR₅R₄, —S(O)R₄, or —S(O)₂R₄, optionally substituted cycloalkyl, optionally substituted heterocycloalkyl, optionally substituted aryl, and optionally substituted heteroaryl;

R₅ is selected from the group consisting of hydrogen and optionally substituted lower alkyl; and

R₆ and R₇ are independently hydrogen or optionally substituted lower alkyl, or R₆ and R₇, in combination with the nitrogen to which they are attached, form an optionally substituted 5-7 membered heterocycloalkyl.

In one embodiment, the Ack1 kinase inhibitor is Vemurafenib (ZELBORAF®), the structure of which is shown below:

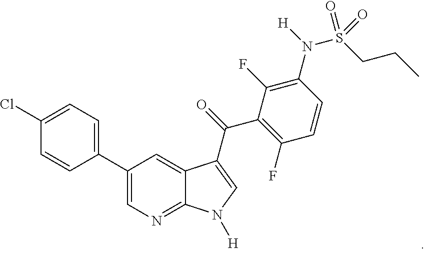 [SMILES: CCCS(=O)(=O)Nc1ccc(F)c(C(=O)c2c[nH]c3ncc(-c4ccc(Cl)cc4)cc23)c1F]

Other suitable Ack1 kinase inhibitors include those described in Farthing, et al. (U.S. Pat. No. 7,358,250); Nunes, et al. (U.S. Pat. No. 7,674,907); Buchanan, et al. (U.S. Pat. No. 7,763,624); Crew, et al. (U.S. Patent Application Publication No. US 2009/0286768); and Salom, et al. (U.S. Pat. No. 8,106,069), all of which are incorporated herein by reference herein for their description of Ack1 kinase inhibitors.

“Lower alkyl” alone or in combination means an alkane-derived radical containing from 1 to 12 carbon atoms, more preferably 1 to 8 carbon atoms, most preferably 1 to 6 carbon atoms (unless specifically defined) that includes a straight chain alkyl or branched alkyl. The straight chain or branched alkyl group is attached at any available point to produce a stable compound. In many embodiments, a lower alkyl is a straight or branched alkyl group containing from 1-6, 1-4, or 1-2, carbon atoms, such as methyl, ethyl, propyl, isopropyl, butyl, t-butyl, and the like. A “substituted lower alkyl” denotes lower alkyl that is independently substituted, unless indicated otherwise, with one or more, preferably 1, 2, 3, 4 or 5, also 1, 2, or 3 substituents, as described below, attached at any available atom to produce a stable compound.

“Substituted” refers to permissible substituents of the compounds and moieties described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups. Furthermore, possible substitutions include subsets of these substitutions, such as are indicated herein. For example “fluoro substituted lower alkyl” denotes a lower alkyl group substituted with one or more fluoro, atoms, such as perfluoroalkyl, where preferably the lower alkyl is substituted with 1, 2, 3, 4 or 5 fluoro atoms, also 1, 2, or 3 fluoro atoms.

While it is understood that substitutions are attached at any available atom to produce a stable compound, when optionally substituted alkyl is an R group of a moiety such as ——OR (e.g. alkoxy), ——SR (e.g. thioalkyl), ——NHR (e.g. alkylamino), ——C(O)NHR, and the like, substitution of the alkyl R group is such that substitution of the alkyl carbon bound to any O, S, or N of the moiety (except where N is a heteroaryl ring atom) excludes substituents that would result in any O, S, or N of the substituent (except where N is a heteroaryl ring atom) being bound to the alkyl carbon bound to any O, S, or N of the moiety.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

As used herein and unless otherwise indicated, “aryl” preferably means mono- or bi-cyclic carbocyclic aryl; “alkenyl” preferably contains 2 to 8 especially 2, 3 or 4 carbon atoms; “alkylene” preferably contains 2 to 4 carbon atoms; “halogen” is preferably chloro or fluoro or bromo; “alkylamino” or “ dialkylamino” preferably contains 1 to 8, especially 1 to 3 or 4 carbon atoms in each alkyl group; and “aralkyl” preferably is mono- or bi-cyclic carbocyclic aryl in the aryl portion and 1 to 4, especially 1 or 2 carbon atoms in the alkyl portion.

The compounds described above may have one or more chiral centers, and thus exist as one or more stereoisomers. Such stereoisomers can exist as a single enantiomer, a mixture of enantiomers, a mixture of diastereomers, or a racemic mixture.

As used herein, the term “stereoisomers” refers to compounds made up of the same atoms having the same bond order but having different three-dimensional arrangements of atoms that are not interchangeable. The three-dimensional structures are called configurations. As used herein, the term “enantiomers” refers to two stereoisomers that are non-superimposable mirror images of one another. As used herein, the term “optical isomer” is equivalent to the term “enantiomer”. As used herein the term “diastereomer” refers to two stereoisomers which are not mirror images but also not superimposable. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981).

In some embodiments, the compounds can be administered as a pharmaceutically acceptable salt of the compounds described above. In some cases, it may be desirable to prepare the salt of a compound described above due to one or more of the salt's advantageous physical properties, such as enhanced stability or a desirable solubility or dissolution profile.

Generally, pharmaceutically acceptable salts can be prepared by reaction of the free acid or base forms of a compound described above with a stoichiometric amount of the appropriate base or acid in water, in an organic solvent, or in a mixture of the two. Generally, non-aqueous media including ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

Suitable pharmaceutically acceptable acid addition salts include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids.

Suitable organic acids generally include, for example, aliphatic, cycloaliphatic, aromatic, araliphatic, heterocyclic, carboxylic, and sulfonic classes of organic acids. Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate.

In some cases, the pharmaceutically acceptable salt may include alkali metal salts, including sodium or potassium salts; alkaline earth metal salts, e.g., calcium or magnesium salts; and salts formed with suitable organic ligands, e.g., quaternary ammonium salts. Base salts can also be formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may also be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibuytl, and diamyl sulfates), long chain halides (e.g., decyl, lauryl, myristyl, and stearyl chlorides, bromides, and iodides), arylalkyl halides (e.g., benzyl and phenethyl bromides), and others.

In some embodiments, the compounds can be administered as a pharmaceutically acceptable prodrug of any of the compounds described above. Prodrugs are compounds that, when metabolized in vivo, undergo conversion to compounds having the desired pharmacological activity. Prodrugs can be prepared by replacing appropriate functionalities present in the compounds described above with “pro-moieties” as described, for example, in H. Bundgaar, Design of Prodrugs (1985). Examples of prodrugs include ester, ether or amide derivatives of the compounds described above, polyethylene glycol derivatives of the compounds described above, N-acyl amine derivatives, dihydropyridine pyridine derivatives, amino-containing derivatives conjugated to polypeptides, 2-hydroxybenzamide derivatives, carbamate derivatives, N-oxides derivatives that are biologically reduced to the active amines, and N-mannich base derivatives. For further discussion of prodrugs, see, for example, Rautio, J. et al. Nature Reviews Drug Discovery. 7:255-270 (2008).

An effective amount of one or more Ack1 kinase inhibitors, or a pharmaceutically acceptable prodrug, salt, or clathrate thereof, can be combined with one or more pharmaceutically acceptable excipients to provide a pharmaceutical formulation. Formulations can be administered to a patient in need thereof to treat cancer, preferably preferably TNBCs. In certain embodiments, the formulations contain an effective amount of one or more Ack1 kinase inhibitors, or a pharmaceutically acceptable prodrug, salt, or clathrate thereof, to treat a TNBC.

Formulations contain an effective amount of one or more Ack1 kinase inhibitors in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

Pharmaceutical formulations may be for administration by oral, parenteral (intramuscular, intraperitoneal, intravenous (IV) or subcutaneous injection), transdermal (either passively or using iontophoresis or electroporation), transmucosal (nasal, vaginal, rectal, or sublingual) routes of administration or using bioerodible inserts and can be formulated in dosage forms appropriate for each route of administration.

The compositions can be formulated for oral delivery. Oral solid dosage forms are known in the art, and described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, K. In: Modern Pharmaceutics Edited by G. S. Banker and C. T. Rhodes Chapter 10, 1979.

In general, the formulation will include a Ack1 kinase inhibitor (or chemically modified form thereof), and optionally inert ingredients which allow for protection against the stomach environment and/or control release of the one or more compounds.

If desired, the compounds may also be formulated as a liquid dosage form for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

If desired, the compositions may be chemically modified so that oral delivery of the derivative is efficacious. Generally, the chemical modification contemplated is the attachment of at least one moiety to the Ack1 kinase inhibitor itself, where said moiety permits (a) inhibition of proteolysis; and (b) uptake into the blood stream from the stomach or intestine. Also desired is the increase in overall stability of the component or components and increase in circulation time in the body. PEGylation is a preferred chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane, and poly-1,3,6-tioxocane. See, for example, Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J. Appl. Biochem. 4:185-189.

For oral formulations, the location of release may be the stomach, the small intestine (the duodenum, the jejunem, or the ileum), or the large intestine. One skilled in the art has available formulations which will not dissolve in the stomach, yet will release the material in the duodenum or elsewhere in the intestine.

If desired, oral dosage forms may contain coatings to protect active agents against the deleterious effects of the acidic environment of the stomach. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (i.e. powder), for liquid forms a soft gelatin shell may be used. The shell material of cachets could be thick starch or other edible paper. For pills, lozenges, molded tablets or tablet triturates, moist massing techniques can be used.

The Ack1 kinase inhibitor (or derivative) can be included in the formulation as fine multiparticulates in the form of granules or pellets of particle size about 1 mm. The formulation of the material for capsule administration could also be as a powder, lightly compressed plugs, or even as tablets.

Colorants and/or flavoring agents may also be included. For example, the composition may be formulated (such as by liposome or microsphere encapsulation) and then further contained within an edible product, such as a refrigerated beverage containing colorants and flavoring agents.

The Ack1 kinase inhibitors may also be formulated for parenteral administration. Preparations for parenteral administration include sterile aqueous or non-aqueous solutions, suspensions, and emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Formulations may also be prepared for transdermal, transmucosal, and pulmonary administration using methods known in the art.

Also disclosed is a method of treating breast, prostate, lung, or pancreatic cancer in a subject that involves administering to the subject PLX4032 (Vemurafenib), or a derivative or analogue thereof.

The disclosed data also demonstrate the potential for “personalized therapeutics” strategy wherein elucidation of Ack1/AKT signaling pathway in a given TNBC biopsy could allow physicians to determine whether that patient is likely to respond to a Ack1 kinase inhibitor. In some embodiments, the method involves first assaying a sample from the subject for Tyr176-phosphorylated-AKT and/or Tyr284-phosphorylated-Ack1. In these embodiments, detection of the phosphorylated AKT and/or Ack1 is an indication that the subject is a suitable candidate for treatment with the Ack1 kinase inhibitor. Compositions and methods for detecting levels of activated Ack1 (pY284-Ack1) and activated AKT (pY176-AKT) are disclosed in International Patent Application No. WO 2010/091354A2, which is incorporated herein by reference for these compositions and methods.

### EXAMPLES

### Example 1

### pTyr284-Ack1 and pTyr176-AKT Expression Correlates Positively with Disease Progression and Negatively with Survival of Breast Cancer Patients

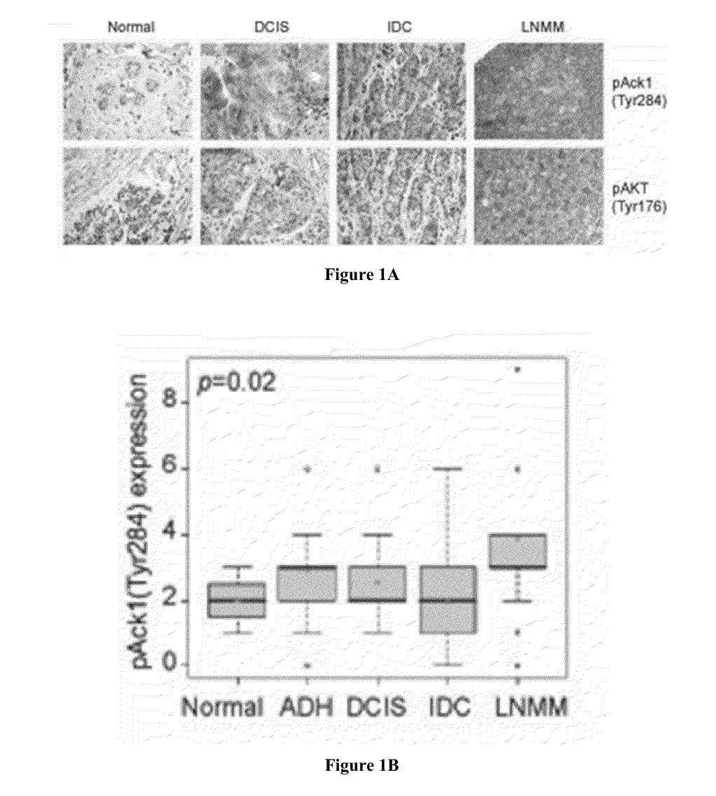

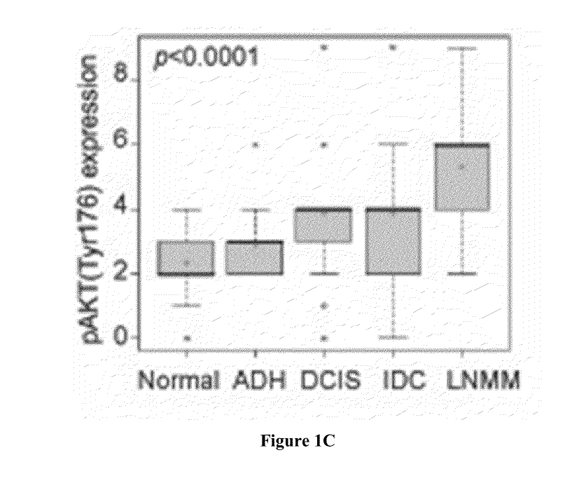

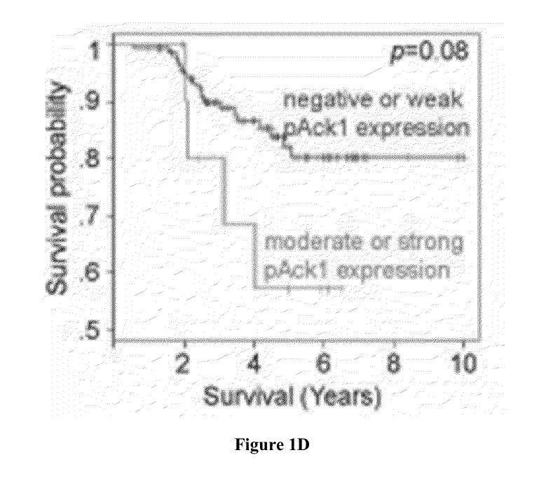

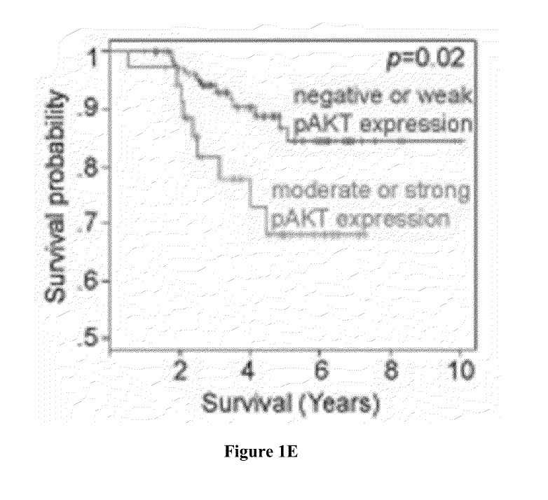

To examine the role of pTyr284-Ack1 and pTyr176-AKT in breast tumor progression, Applicants performed an extensive tissue microarray analysis of clinically annotated breast (n=476) tumor samples. Immunohistochemical analysis revealed significant increase in expression of pTyr284-Ack1 and pTyr176-AKT when breast cancers from progressive stages were examined, i.e. normal to hyperplasia (ADH), ductal carcinoma in situ (DCIS), invasive ductal carcinoma (IDC) and lymph node metastatic (LNMM) stages (FIG. 1A-C). pTyr284-Ack1 and pTyr176-AKT expression correlated positively with the disease progression and negatively with survival of breast cancer patients. In contrast to pTyr284-Ack1, the total Ack1 levels remained unchanged between normal and tumor samples. Kaplan-Meir analyses revealed that patients with high expression of pTyr284-Ack1 and pTyr176-AKT are at a higher risk for cancer-related deaths (FIG. 1D, E). Furthermore, expression of pTyr284-Ack1 was significantly correlated with pTyr176-AKT (Spearman rank correlation coefficient p=0.43, p<0.0001).

### Example 2

### PLX4032 (Vemurafenib) and AIM-100 Inhibits Ack1 Activation

Applicants sequenced ACK1/TNK2 exons in primary tumors using second generation sequencing approach and identified three novel somatic mutations (Unpublished data). Site directed mutagenesis was performed and HA-tagged point mutants were generated. HEK293 cells were transfected, grown in growth factor free media, followed by immunoprecipitation with HA-beads. Immunoblotting with pTyr antibodies revealed that WT Ack1 construct exhibit modest autoactivation, in contrast, Ack1-F224L point mutation is highly autoactivating (FIG. 2, top panel). Other two mutants, Ack1-K291E and Ack1-1038C exhibited moderate autoactivtion (FIG. 2). To assess whether Ack1 activation is sensitive to Ack1 specific small molecule inhibitors, cells were treated with 5 uM of AIM-100 or PLX4032 overnight. Both inhibitors completely abrogated Ack1 activation (FIG. 2, middle panel).

### Example 3

### AIM-100 Inhibits Ack1 and AKT Activation

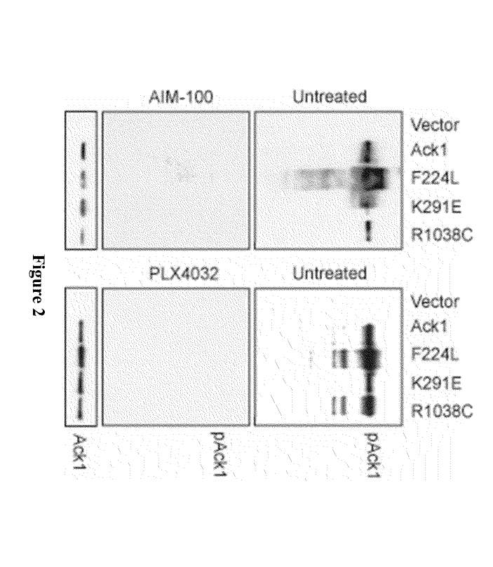

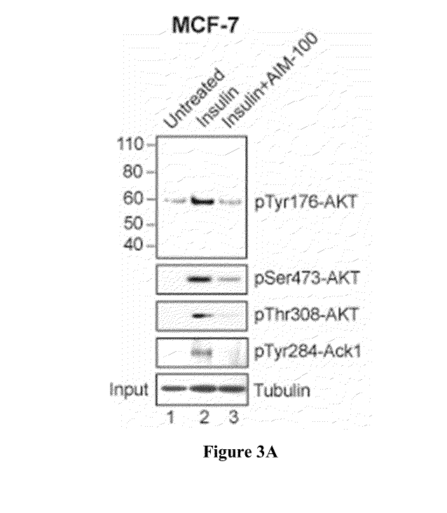

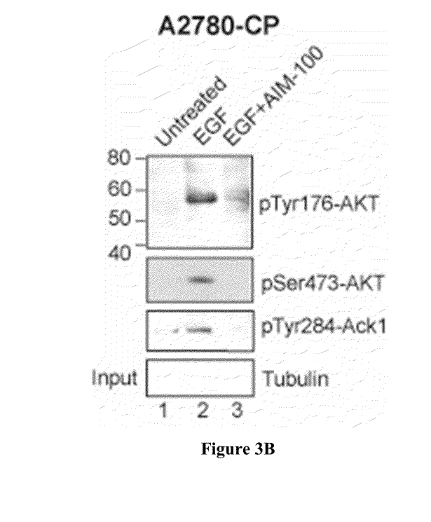

To assess the potential inhibition of Ack1 activity, MCF-7 (breast) and A2780-CP (Ovary) cells were treated with AIM-100 overnight. Cells were either untreated or treated with Insulin (for 30 mins) or EGF (15 min) followed by immunoblotting of lysates with pTyr284-Ack1 antibody. Insulin/EGF treatment resulted in significant increase in Ack1 activation, as seen by increase in endogenous pTyr284-Ack1 levels, which was significantly decreased upon AIM-100 treatment (FIGS. 3A and B). Further, insulin/EGF treatment resulted in significant increase in endogenous pTyr176-AKT levels; however, AIM-100 treatment resulted in significant decrease in AKT Tyr176/Ser473 and Thr308-phosphorylations (FIG. 3). Collectively, these data indicates that AIM-100 can not only inhibit Ack1 activation, but can also inhibit AKT Tyr176-phosphorylation and subsequent activation.

### Example 4

### PLX4032 and AIM-100 Inhibits TNBC Derived Cancer Cell Proliferation

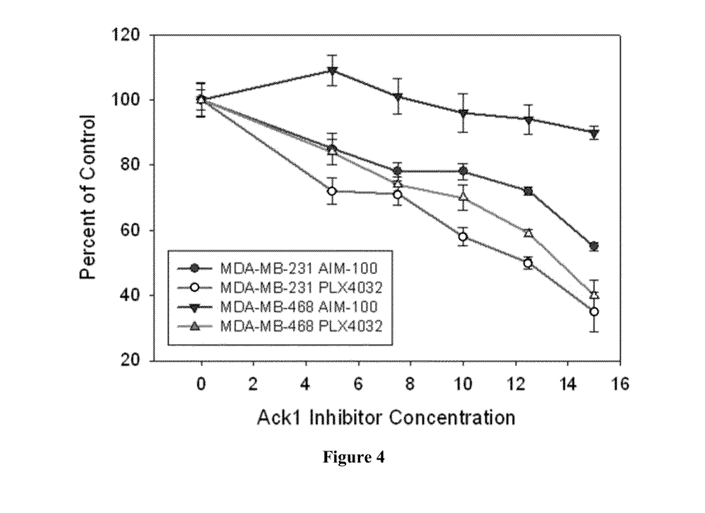

To determine whether inhibition of the Ack1/AKT signaling pathway affects TNBC-derived cancer cell proliferation, MDA-MB-231 and MDA-MB-468 cell lines were treated with increasing concentrations of PLX-4032 or AIM-100. A cell growth assay was performed by adding solution of WST1 to 96 wells (5×103 cells) and incubation for 1 h. The cell viability as a function of mitochondrial activity in living cells was measured spectrophotometrically at a wavelength of 450 nm. Both TNBC derived cell lines exhibited significant decreases in cell growth upon treatment with PLX-4032 with IG50 of ˜13 μM for these cell lines (FIG. 4). Interestingly, AIM-100 inhibited MDA-MB-231 with IG50 of ˜16 μM but MDA-MB-468 cell line was rather resistant to AIM-100 mediated cellular cytotoxicity.

### Example 5

### Affinity Purification Coupled ELISA

Step I. In vitro pTyr176-AKT Detection

Rapid and accurate detection of pTyr176-AKT in cancer biopsies is an urgent need for TNBC patients. Towards gola of developing a reliable diagnostic system, we first designed and tested an ELISA assay that recapitulates Ack1/AKT signaling nexus in vitro. Two peptides derived from AKT were synthesized that were biotinylated at carboxy terminus (shown below). The peptides sequence is derived from the region that is recognized by Ack1 leading to AKT Tyr176-phosphorylation and activation (13, 15, 17). The phospho-AKT peptide was used as a positive control and as a standard for quantitation of pT176-AKT in tissue samples.

AKT peptide: VKEKATGRYYAMKILKKEVI-biotin

pAKT peptide: VKEKATGRYpYAMKILKKEVI-biotin

The AKT peptides were diluted to final concentration of 1 uM in phosphate buffered saline (PBS) and immobilized onto streptavidin plates (R & D systems) for 1 hour. Unbound peptides were washed and AIM-100 or PLX4032 (50 nM) suspended in reaction buffer (10 mM HEPES, 20 mM MgCl2, 75 mM NaCl, 0.125 Twin-20) were added followed by purified Ack1 kinase (50 nM) with 1 mM DTT and 5 uM of ATP. After 1 hour, plates was washed with PBS containing 0.1% Twin-20 (PBST), blocked with 3% BSA and pY176-AKT antibodies were added (1:100 dilution in BSA). After 1 hour of incubation, plates were washed in PBST and secondary antibody (HRP-conjugated anti-rabbit antibody diluted in BSA) was added. After 1 hour, plates were washed in PBST and devoped using OPD substrate (Sigma) and read at 450 nm.

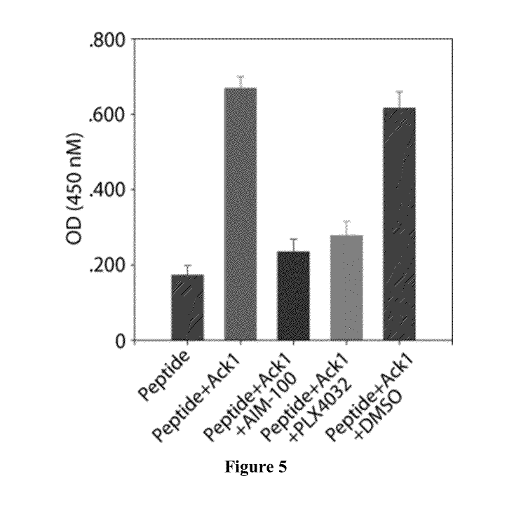

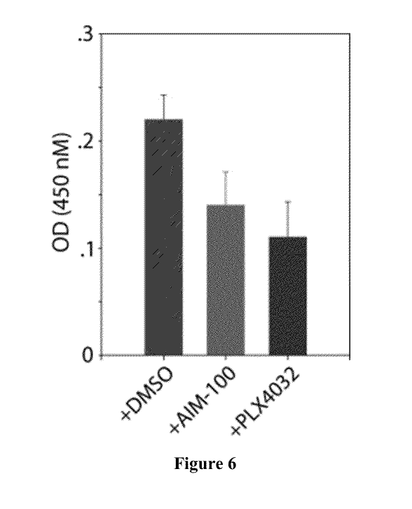

Incubation of AKT derived peptide with Ack1 resulted in robust AKT Tyr176-phosphorylation which was detected by pY176-KAT antibodies (FIG. 5). In addition to using pY176-AKT antibodies, we have also used pTyr-antibodies that are HRP conjugated (Santacruz) and very similar data was obtained (data not shown). Upon incubation of thie reaction with Ack1 inhibitors, AIM-100 and PLX4032, significant decrease in AKT Tyr176-phosphorylation was seen (FIG. 5). Collectively, these data establishes rapid AKT Tyr176-phosphorylation detection method. Further, it indicates that loss of Ack1 activation by AIM-100 or PLX4032 can be efficiently detected by assessing pY176-AKT levels.

Step II. In vivo pTyr176-AKT Detection

Towards detecting pY176-AKT in TNBC derived cell lines, MDA-MB-231 (1×104 cells) were lysed in reaction buffer containing phosphatase and protease inhibitors and lysates were quantitated. Biotinylated AKT peptide was incubated with streptavidin beads for 1 hours, beads were washed and the AKT peptide coated beads were incubated with cell lysates for 1 hours at room temperature. The beads were washed in PBST and the bound peptides were eluted. To elute the bound biotinylated AKT peptide from Streptavidin Beads, Streptavidin Beads were resuspended in 10 mM NaCl containing solution followed by heating the mix at 75° C. for 2-3 minutes, as described in literature (33). The eluted AKT peptides were immobilized onto streptaviding plates and presence of phospho-peptides was detected by ELISA described above in step I.

A significant decrease in pTyr176-AKT levels was observed in cells that were treated with AIM-100 or PLX-4032, suggesting that Affinity Purification Coupled ELISA can detect pTyr176-AKT in vivo in TNBC-derived cell lines. Further, these data confirm sensitization of MDA-MB-231 cells to PLX-4032.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for treating Triple Negative Breast Cancer (TNBC) in a subject, comprising administering to the subject a composition comprising a Ack1 kinase inhibitor.

2. A method for selecting a therapy for a subject with Triple Negative Breast Cancer (TNBC), comprising
(a) assaying a sample from the subject for expression level of Tyrosine 176-phosphorylated-AKT, Tyrosine 284-phosphorylated-Ack1, or a combination thereof; and
(b) comparing the expression level to a control level;
wherein detection of an elevated expression of Tyrosine 176-phosphorylated-AKT, Tyrosine 284-phosphorylated-Ack1, or a combination thereof, compared to the control is an indication that an Ack1 kinase inhibitor is selected as the therapy for treating the subject.

3. A method for treating a subject with Triple Negative Breast Cancer (TNBC), comprising
(a) assaying a sample from the subject for expression level of Tyrosine 176-phosphorylated-AKT, Tyrosine 284-phosphorylated-Ack1, or a combination thereof;
(b) detecting elevated expression of Tyrosine 176-phosphorylated-AKT, Tyrosine 284-phosphorylated-Ack1, or a combination thereof, compared to a control level; and
(c) administering to the subject a composition comprising a Ack1 kinase inhibitor.

4. The method of any one of claims 1 to 3, wherein the Ack1 kinase inhibitor is defined by Formula I 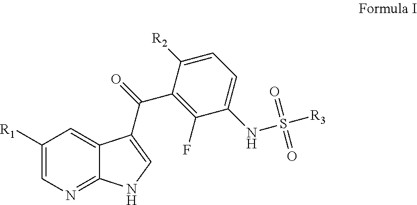
or a pharmaceutically acceptable salt or prodrug thereof,
wherein
R₁ is selected from the group consisting of hydrogen, halogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —OH, —NH₂, —CN, —NO₂, —C(O)OH, —S(O)₂NH₂, —C(O)NH₂, —C(S)NH₂, —NHC(O)NH₂, —NHC(S)NH₂, —NHS(O)₂NH₂, —OR₄, —SR₄, —NR₄R₅, —C(O)R₄, —C(S)R₄, —C(O)OR₄, —C(O)NR₄R₅, —C(S)NR₄R₅, —S(O)₂NR₄R₅, —NR₅C(O)R₄, —NR₅C(S)R₄, —NR₅S(O)₂R₄, —NR₅C(O)NH₂, —NR₅C(O)NR₅R₄, —NR₅C(S)NH₂, —NR₅C(S)NR₅R₄, —NR₅S(O)₂NH₂, —NR₅S(O)₂NR₅R₄, —S(O)R₄, and —S(O)₂R₄;
R₂ is selected from the group consisting of hydrogen, fluoro and chloro;
R₃ is selected from the group consisting of optionally substituted C₂-C₆ alkyl, optionally substituted aryl, optionally substituted heteroaryl, and —NR₆R₇;
R₄ is selected from the group consisting of optionally substituted lower alkyl, optionally substituted lower alkenyl, provided, however, that when R₄ is optionally substituted lower alkenyl, no alkene carbon thereof is bound to N, S, O, S(O), S(O)₂, C(O) or C(S) of —OR₄, SR₄, —NR₄R₅, —C(O)R₄, —C(S)R₄, —C(O)OR₄, —C(O)NR₄R₅, —C(S)NR₄R₅, —S(O)₂NR₄R₅, —NR₅C(O)R₄, —NR₅C(S)R₄, —NR₅S(O)₂R₄, —NR₅C(O)NH₂, —NR₅C(O)NR₅R₄, —NR₅C(S)NH₂, —NR₅C(S)NR₅R₄, —NR₅S(O)₂NH₂, —NR₅S(O)₂NR₅R₄, —S(O)R₄, or —S(O)₂R₄, optionally substituted lower alkynyl, provided, however, that when R₄ is optionally substituted lower alkynyl, no alkyne carbon thereof is bound to N, S, O, S(O), S(O)₂, C(O) or C(S) of —OR₄, —SR₄, —NR₄R₅, —C(O)R₄, —C(S)R₄, —C(O)OR₄, —C(O)NR₄R₅, —C(S)NR₄R₅, —S(O)₂NR₄R₅, —NR₅C(O)R₄, —NR₅C(S)R₄, —NR₅S(O)₂R₄, —NR₅C(O)NH₂, —NR₅C(O)NR₅R₄, —NR₅C(S)NH₂, —NR₅C(S)NR₅R₄, —NR₅S(O)₂NH₂, —NR₅S(O)₂NR₅R₄, —S(O)R₄, or —S(O)₂R₄, optionally substituted cycloalkyl, optionally substituted heterocycloalkyl, optionally substituted aryl, and optionally substituted heteroaryl;
R₅ is selected from the group consisting of hydrogen and optionally substituted lower alkyl; and
R₆ and R₇ are independently hydrogen or optionally substituted lower alkyl, or R₆ and R₇, in combination with the nitrogen to which they are attached, form an optionally substituted 5-7 membered heterocycloalkyl.

5. The method of any one of claims 1 to 3, wherein the Ack1 kinase inhibitor is Vemurafenib.

6. The method of claim 4, wherein the Ack1 kinase inhibitor is Vemurafenib.
